SEQUENCE LISTING (9 sequences)

SEQ ID NO: 1 - DNA, 20 bp, Artificial Sequence
  It is synthesized
caggctgggcaacatagtga

SEQ ID NO: 2 - DNA, 19 bp, Artificial Sequence
  It is synthesized
cctgggctcaaacgatgct

SEQ ID NO: 3 - DNA, 19 bp, Artificial Sequence
  It is synthesized.
ttgatggctggcagtgctc

SEQ ID NO: 4 - DNA, 20 bp, Artificial Sequence
  It is synthesized
ccatgttgaggcagcacatc

SEQ ID NO: 5 - DNA, 19 bp, Artificial Sequence
  It is synthesized.
ggccagtgaccttgacctt

SEQ ID NO: 6 - DNA, 20 bp, Artificial Sequence
  It is synthesized.
ttgctgcctcttatgctcac

SEQ ID NO: 7 - DNA, 7619 bp, Homo sapiens
ggggaggagagaaaaactgtgggacctagtgaaggatgtaggagaaagttgttatcattgttttagcaataggagaattgtgtgcatgtttttggtgctaatggaaaggacccgagataaaataattgagggaagagatgggagccagggcacgatagagccttcattaagagggatgccctttctaagaggaggaaaaatggatgcaagcatagttgtcatggttttgatagagaacctcatgtggctgtccatcaaacatcacttgagtcataggtgtctagaacactattttgcaccttagaaatatttgaatagacaccttagaaatctttgctgagtaagacagattcctcgcatagctgcaagaagcttagccaaacccatttctaaattaaaactaccagcaaagaggtacttgagtaataggcacagggtgctcagctctgggctaatagcattgaatgtcctgatttttagaatgttctgtgcttaacttctcaggcaacacaggctgaagtataagagttgaagggatttagaataagtcattgtttcattaaaaattatttttggctgaaggagaaataagatttggatggggacacagctaaatcatatcatataccatcttaattggggatggtattttttttttttcttgagacagagtctttctcatgctattctcgtgatagtgaataagtctcatgagatctgatggttttataaagggatcttctcctgtacatgctgtcttgcctgccaccatgtaagacatgcctttgctcttccttcacattctgccatgattgtgaggccttccccagccatatggaactgtgagtccattaaacctctttcctttataaattacccagtctgtccattaaacctctttcctttataaattatccagtctcaggtgtgtttttattagcagcatgaaaatggactaatacagtgtataagccccaaattctaaccacctccttgagtcacatttccttgtgaatttccatgccaaccaacataattaaatgttgctgtctcttgtgaatctgtcttttatcagtttaatttacagggctccaagaaaagaaccaaagagggcagaggaacagtttttcctctctgacagaataaaagggcatgatgtttgaagcttacttccaaatggttctgagagaatgagctaatagagcaaatgtagcaaaacattaacaattgttgaatctgggtgaaggatttgagggagtttttggtactgttcttgaaacttttttttgaaattatttcaaaataaaattatttttaaaagaagaataagcttctgcatttattcatcacctctgctttttttttttactctatttccactttcaacacttttttactcacaacttcctatgagtggaaaggatgaggaatttgaaagacagggctgccttttgggtttgtgcaaaatctctgtgtcttaatttgtctatctgtaaaatgaggataatatcttcaaacgtttgctacatgaactgagatgaggatacagtaggccctcaataaatgttattgcctacccttctaagaccagttcaagtcctatttcctttattaagtcttccctcatcacatccatgataaaaatttattttatatcgtaccatacatttagctaaatggctttctcatacattacctcattgaattatcccaataaccttacaagttaggttatcattttctctacatcatagataaggacatttaatgaagttaaaagatttggctaaaatcacacagtcggtcaatagaaataacagaatgaaaactcaggttcttttagccccaagttgtatcctctatgttataccacaatttgatatttctcctactcaagtgttttaagtgtggtaagaatatacataacataaaatttactatgttaacaattttaaaatgcactgtttagtggcattaagtactttcacgttgttatgcaaccactatccatcactagaactttttcatcttgcaaaaactgaaattctgtaccatttcaacaccaactccccactcctcccttccccccagcccctggtaaccaccattctactttctgtctctatgaatttgactgctgtagatataagtggaatcaggcctggtgtggtggctcacacctgtaattgcagcactctgggaggccaaggcgggagggttgcttgagctcaggagtttgagaccaggctgggcaacatagtgagacctgttgtctacaaaaaaattaaaaaattagctggaagtggtggtgcatacctgtagtcccagctacttgggaggctaaggcaggagcatcgtttgagcccaggaggtcaaggctgcagtgagctgtgatggcgccactgcactccagtctgggcaacagagcaagaccctgcctcaaaataaaataaaataggccaggcgtggtggctcacacctgtaatcccagcattttgggaggccgaggtgggtggatcacctgaggtcagatgtttgaccaacatgctgaaaccccatctctattagaaatacaaaaaaaaaattatccgggtgtggtggcgggagtctgtagtctcagctattcgggaggatgaggcaagagaatcgcttgaacctaggaagtgcaggttgaagtgagccaaaatcaggctacagcactccagcctgggcaacaagagcaaaactccatctcaaaaaataaataaataaaataaaataaaaatttaaaactaaaaaataattatacaaaaagggaatcatacagtatttgtccttttgtgattggctgattccacttagcacgatgtcctcaaggttcatttatgctgtagcatgtgtcttaagttttataaacacttgtgctcatggcactcatgtacaacttcgtgtgtccttattttctttcctggtttataaactccttgggggctgaaatcccatgctttctcttggtattgccacgctcatagtgcctcgttcataacagtgcccaatgaactctcttgaatgggtagatacaggccactctaaagtagaaaacgatccctctcctatacttgatgaaacttgtatacagtttcatcactgctgtcagctattggagacaaggagctggacaagcataattgttctccctactattgaaacgggagccagacgaattaagaagattccccaggttggaagtatgacaggcaggtgaactttagtcaagtagaattagctctctgcccagtgtaaaaacgtatatgcagtattcttcaaagaggaaaaaatctcatcttttagtcatatgaagtgagagagagtttggtcgtggcttttttttttttttttttttccctgagaaaaagaaatgagatctgtcaagactgaactttgcaggagagaaatcatcaatctttggtgtcatctgagtgcattggaaatgcatttgccttgtcttgcggtttgattgttgtctaatccctgtaaacattaaaggataaaaacaggagaaatggaacaagaaatatgtgggatccaggttttagctgtgctgcccgctggtgactttcgaggtgaagctttcaggtcccttctccctagcacacccatgcctccacctctatgaagcaatagagcttcaagggcaaatctggcctcagagcccagaggggaatatggccacatcagggaccaccccaggagactcaaaggcctgcatctctttgtcttgcaggagttgctgctggaccacatgacaccagtcccattcttgaagcagaatgttcaaagagtgtgtacaaaatgcagaagccacccatttccaagaaaaacggctatgcttcaaccagcctaagtctactgcaagcaagggcacccataagagatggaccttgagggattcaacctaactaactgtgagtaaggacgtctagcacattttggagagtactggtatgagacagattccaaataaaataaccggaggcaggctgggtgcagtggctcatgcctataatcccagctcttagggaggcagaggcaggaggatagcttgagcccaggagttcgagacttgcctgggcaatgtagcaagaccccattctctacaaaaaggaaaaaaacaaacaaacaacaacaacaaaaaaaaaaaaacaagaaataactctaggcagcctgtaacatactaaattatttcccattagcacattagcacataaacaccattagcacatctttttgaatatgtaaggtctgtggagataagactacacacaggatctcaaagaatctcccttactaagggggaaatcaagttggcttttatcctagcaacttacttaagccttaacatacttacaattgttttacatagtatataatatttcccatacttgattgtgtttcttaagtttcatttttaattttcaaatcaaaacaggaaatatgggctgggcacagtgtcccacactctcagcactttgggaggatagcttgaggccaagagatcaagaccagtctgggcaacatagcaagactctgcctctacaaaatttttttaattagccaagcatggtggcatgcatctgtagtcccagctactcgggaggctgaggcaggattgcctgagccctggagttcgatactgcagtgagctaggatcataccaacatgctccagcctgagtgagagaggaggaccccgtctcaaaaaactgtcaaaaaaaaaaaaaaaaacagaaaatatagagaagaggtagctatttagaatgcttgtagaacttttgtaacattactgttttttagactgaggaacattctccaaaaatatgattttgcacacacttttgattgcctaaccaaaagtcatttctctctctctttcccttctggcagggcctgcttcccataagaagggctaaaaatgccagacactctctttcagcctcttttgcaggtagggctggccacatgacccatctggtcaaattaacctttcagccacaccctcccaaaaggcttttagaaagatttttcatggtaatgaaaacaacagatattcaaggagagctttttttccccacctcttccttcctgacatttcataggcatttcatttcatgggagctatggaagctatattgcagccattaagcagtagacaaagcgaacgtgctgaggatggcaacaaacagggatggaaagaacctggatcactgatgatgacactgtttagcttgggaccatcaccttctgtattagtcagggttctctagagggagagaactaataagatttatatatatatacataaaggggagttaattaagtattaactcacacaatcaagaggtcccacaacaggcggtctgcaatctgaggagaaaggagagccagtccgagtcccaaaactgaagaactgggagtccgatgtttgagggcaggaagcatccaacacgggagaaggatgtaggctgggaggctaggcaagtctagtcttttcacatttttctgcctgctttatattctacccatgctggcagctgattagatggtgcccacccagattaagggtgggtctgcctttcccagtccactgactcaaatgttaatctcctttggcaacaccctcacagacacactcaggatcaatgctttgcatccttcaatccaatcaagttgacactcagtattaaccatcacacgttctaaacaccttattatgtaaaatgatacaatcctgttggatatgccacctttttttgaaacagggtctcaccctgttgcccaggctacagtgcaatggcatgatcacggctcactgcagcttcaaactcctgggctcaaagtgatcctcccatgtcagcctcctgagtagctggaaccacaggcatgtgccaccatgcctagctaattttttttttagagagagacaggctcttgctatgttgcccaggctggtctcgaactcctgggctcaagtgatcctcctgcctcagcatcccaaactcctgggattttaggcatgtggagccattgtgcgtggctggatattgtttttaccacttttagctggatattgttattcacagctgaaagcaccttaacatattcaatgcgattttaaatacagtccaagtggaacctattttacaccaaatattgactaactttctgtcagacattggaaggccatgttcctaaacataagcatatgagaaaatccttgtaatgatgagataaagggagtttgccagtttaaacacccgcgggttgccctggcaaatgtcaggtgtcacagggtcggaaagattagttcataatgtactaggaaaaacatcctgcaatgtatatgaaaatatatattgtaaaaaatctttttggctaaatgtattggcacgattattagaatacaatctagaaacatttactttaaattttaagagcaaagacaagacttctaatcttaattagtcaaagtttttcttaaggatttcaattcaaatgtgtcctttattctgattgtgatgggacttttgatttctcctatttgaaatgccatgtgaatgtcttagaaaacccagacatctgaccttatattttacttgataaattgaaaaagccacttgctgataaaattttgttattcacaggtattacatgagtgaattcatttttctacttgctagttttctagacaactatcttcattaaaattttagattactgtaatttacatggagttcacacattcaatttacagatttagaatacttaatcaattgatgtccattttattttttcatttagttgctgagataagacatccatttttctcaaaatgtatcagatcaagctaaaaaaaaagaaatgaaattaatatgaaaatatctattaaaggacatcagtgtccaagctcaccatcctttctaactatctttcatctgaacatagtcagggcttatacaatagaattaaatataccatcccagaggacacctggcagtgtccagagacatttttggttgtcacaacatggaggcgcagtgccactggcatccttggtagaggacaaggatgctgctacacatcccacagtgcacaggacaacccccacagcaaagaatgctccagcccaaaatgtcaatagtgccagggttaagaaaccctgttctacaccaagcaccttgatgtctaaaatacaacacaaactggttctttgttcattagaattcctacagaggaaagaatagattactatcattttacagtcatctcaacaattgaagtcaggagaagccttgattcttataaactgcaaaacattagtttgaagtctagtttgtaggtatcatttgttttctatgaggaattccttcttttgatgttatattttctttcattcaaagacaggacttacatgagccttcctttctgctgttccttttcttaaaaaaaaatgtcttatggctgggtgcagtggctcatgcccgtaatctcagcactttgggaggccaaggcaggcggatcacctgaggtcaggagtttgagaccagcctgcccaacatggtgaaaccctgtctctacgaaaaa

SEQ ID NO: 8 - DNA, 5168 bp, Homo sapiens
cccgcaggcatcgggccaatctcagagggcgcgcacgccgcatcaggaccccggaacccgccccgttggggatagggacagatggagaatccagctcagacccagcaactctgagctgacttggatctaattcttcccaattcatgccacaagcccaactcatcagctgatacctgaggatgatccactgctcactgagcgtggtcagggagcgccgctgtcggacgagcatcttcatcaaatgtgctgagaaccctctgcacctctccacgttgcccatgcccatttcctggcagaagaaggaaaaacatgctgaaggggctgctgacaaacatcacccacagatgtgcctctagaaagctgtttctactataagcgtcagacatgccacactctagagatttctgttcagcaaagaccagagctcactctatcccaaacacaggactgtgtgtcagtgaacaagtaaaacaagtctaaaaaagaaaccatgggctaggcgcggtggctcacacctgtaatcccagcactttgggaggccaaggtgggcggaatcacaaggggggaacgaagttccacaattttggcctggacccggtcaaagaattgcttgtaataatggtacaaattccatagaacactgcaaagtgaatctggaagaaatgaaaaggaaacaaaaataccacacttgctgcatgtgctactggtgccagctgtggccttcatatttcagtgatgacccttcctcattactctctatgccatgagattacagtgagtttcacaggtgcattatttagggatcctgtctgagtttagcatttcccagagatgcaggctgtcaacactcccctatttttcacttctgcttgtaacagatgtttttttgcacttttgttcttgaagatgatgtactcctaggttcttctaaaatcacaatgtcaaattccttctgtgaaacttattgtaaaatcagcagaaactgagctttttttaaaagatggcatattaagcagttcacctcaaatgctcattcatgtacaatctatactcatcttgtagtgagacttttatcccagttgctgtgatcagggatagtacttgacagcgggcagttttagatgcagttccttgatggctggcagtgctctcactggatgaaagaaactaataatggcctggatcaaggagcagcttagcaggacatttacaatgtctagacagagagatgtgctgcctcaacatggtaggtcctgagatcttcatctggcaatttacctctgccagagctcactagtcagtactaaacttataaccatctgcacctcactttgaatgttcttctctatatctaatgtgaatcgcattaaacataactaaatgtgtcttgtttaaaacctagcgtgcaacaaggcacctgtctcatgcagagaattcaaagcctaactcattttagtcgtgcaatgtcaaaggaaaggaaaaaaaaatcctcaccccttccttcaacctgtggcatcagcaagacatgacaatggaaaaccagtaacatctgaagtcgcacatggaactctcccagcgaggatccttcaataaatgcttgtaatgtgctgaccagcaacatcaaggtcatctgtttgtcatctttaaaaattcaaagagaaaaatatatatgttccttcgaccatgactccctattcctaacatgcacgcaatctatttgggatactacggatggccaagaggtagacatcacaatgtgaagctctcaagcaaggaccatggctaaattctaagctgatttaaaagtaacaggccggtgtggtggctcacgcctgtaatcccagcactctgggaggccaaggcgggcagatcatttgaggtcaggagttcaaaaccagccttgccaacatggtgaaacctcgtgtctactaaaaatacaaaaattagacaggcatggtgttgggcaccggtaatcccagctactgagaaggctgaagtaggagagtcacttgagcctgggaggcggagattgcagggagctgagatcgtaccactgtactccagcctgggcaacagagcaggactctgtctcaaaaaaaaataaaaaacaaatataaataaataaataaaagtagcagctgcacatagagcatgtgttaaatgcactcacagcaaattatgaaaatgaattataaggtgcttgtcacaagtataaaataaaagactgttcattcaagcatgttggctactacctaccaaaaagtcagctttcagtttgacttctatcatggacatagagaagacggaaaccttatcctacgactggttctctagacttttggagaagacatttaaacctctcacacttttgttattattctctcagttagataagcacgatttgcagttttcttggctgacctctcagaaatatcatgaagtcaaatgggaggcattcccatgaaagccatgtagcgggagaagcgggagcaggaaaatcctacactttgcagcactccatttagccgggaagctctaagtgggcaggaaatgtactgctaggcattaccttcctgttcttctgtttgttcctgcatgtgcttctcaagcatctgatagatggagaaccagtgcttggtggatttctcggtgtggcgcttcatagtattatccaaactcatggaccagcagctgaaacgagaagaagccaaggaggcatttaggaaaacctatttgaaatatctgaaaccaaagatgcagaagctacatgctcacatttctctcctcccaaatggagatggaggagccacccagtttcctccctcaagaatatatgctgttgtctggactgtattcaggtcattttaatgccctacttttaactctgtgattaataactgaaaagcacattctgtagctgtaatactatataaatcctaaagaaatttaaactaccaaaagacaaaagcccttacaacctaagtttaggagagggaaggagagtcacttacttcagctccagtttacgccaccgaatgatcatctgactgatcaaatcaagatgtttccgcaaagacaaagctcgacttgcattttcctcccaatcctaaagaaacaaagataaaatttactacagatattattcaatgccataatcacctccttcctcaagaacatcttgaactttgcttgagtgaagactactgggcccttaaacaacttgcaccaattgtaggaatgaatttacagtgtatcagcttcaagttctttctgttcacatgacagaactccaccagagccaagtttagaggctggcaaaaaagcacagtgtttatagactggagcctgaattcgtgctctttgctatctgacctagaacaaagacatgcggcccttttctgtccaatggggaggcaacagctaacttagaggccatgagggcactgaggggaaccgtgtatgtgggcactcagttggaatgaaggcccacctggaaggctgggaggagtgcaccctcattaccggtagatactgcatcccactggataaaaggcaaggcagctagccctcaccacaggggcaacagcctcacttgcaatgatctcaccccatgccagaaaaaccaaggatgtttacctttttctagcccaaactataattttctgtctaacaacaaaggtgccacaagacgtatcttttgccaactgaccaccaactaaaatgaggaagagaggtattagctcacctgtgcctttgccagaaggatctctaagccattcaggaactttgagatgggactggaaagtgggaaactacgaattctgtccattacaaccaggagctgcagaaataaagatttgggtatctcagtaggaataccagtgatcctaagtcacagggtactgaagagagtcctgaccagaagattccccccagttctctacttttaaaacacctctgaaaaaaaaaacagagcaacatggcagcattttattatgtggtcagcaactcctgaccaacaaataaaattaacagaatccccaatcatttacacaagagaaatacaaaactggtgtgttcatcctgcagttagtaagtaacatctcattgaactgcagttctttaagcctttgtttatatgtatatatgtgcaagtgggtgcccccagggataatggtgaggctgcatcctcattgcagactcagtactgtgagcactccttcctcaacagctgccctacctgttcaagcgctgggtgttctggccagtcctgtagcaagtgactgacagcctctgagaaaccttgaagcacaggttgacactgccgtgcttctggaacattgtgatgctggtagaagtcatagggcccatcaggtttcaccatcaggtctgagggtgcctccccaaaaagagtgttatgggagagggtacaggccaaaagttggctgcccaagagtcggtcattcagttcaactcctgtgaggttaacatcacggtaaagtcagtgaattcagtaagttgatctatcaacatgtcaaaaagatgtcagcaacagggccaggtgcagtggctcatgtctgtaatcccagcactttggggagccgaggcaggcagattgcttgaggccaggagtttgagaccatcctggccaacatggtgaaacctcttttctactaaaaatacaaacattagccagacatgcatgtgcctgtaatcccagcttcttgcgggcctgaggcacaagaattgcttgaacctgggaggcagagattgcagtgagctgagattgtgccactgcactccagcctgagaaacagagcatcagcaacatcagcaacaatgaactaattgggactaatgaatgctattctcccctaaagcaacattggcctagagaactgtaatcctaatactatagtagctacctaaagctctcaagtttctggaatgtgcagaggaagtcagaaactgcctaggtataaacaaatggtgtaggggttaactcagtgcatccaaattcccagtttttatacctattctaggtaaacagaaaggaaacttaaagcctccttcctacttctatgaaataatgccaagaaatcatgtaggaaactgcaatgaaccaactattaaaccatattctacaagaaaaaaaaaaaaaaaaaaaa

SEQ ID NO: 9 - DNA, 432 bp, Homo sapiens
acatgtcaacaacatcaacaaatagaaagcattcctccattcatcactccttctgggaagaagtagactaagaggtaagaagcaactaatttttcatgggatttgaaactaagtaggtgaatctgaagtctgcgctgcttccattccatcccattgtctcttctgagggagaaagcttctaagggagaaagcctttggggcttgctggtgatctttttctacacaaaaagttttcaaacaaaaggaacaaaagaagccagagtctttagagtacacactccgtacttccattaaataaatttcaacaacaggtcaaagcatgtatcttgcaaccttctttgaataaggctcagtgcaaagctggggccagtgaccttgaccttccttcatccctatctatcaaggattttttgtgagcataagaggcagcaa

(19) United States
(12) Patent Application Publication
(10) Pub. No.: US 2020/0270690 A1
(43) Pub. Date: Aug. 27, 2020

(54) Serum/plasma LncRNA marker composition associated with auxiliary diagnosis of intrahepatic cholestasis of pregnancy and application thereof

(71) Applicant: Wuxi Maternity and Child Health Care Hospital, Wuxi (CN)

(72) Inventors: Ting ZHANG, Wuxi (CN); Liang LUO, Wuxi (CN); Jing WANG, Wuxi (CN); Ruirui DONG, Wuxi (CN); Ping ZOU, Wuxi (CN); Shaohan ZOU, Wuxi (CN); Rui YANG, Wuxi (CN); Yao WANG, Wuxi (CN); Na LI, Wuxi (CN); Minjian CHEN, Wuxi (CN); Xina MENG, Wuxi (CN); Yan ZHANG, Wuxi (CN); Conghua ZHOU, Wuxi (CN)

(21) Appl. No.: 16/781,973

(22) Filed: Feb. 4, 2020

(30) Foreign Application Priority Data
Feb. 22, 2019 (CN) 201910132726.3

(51) Int. Cl. C12Q 1/6883; C12Q 1/686

(52) U.S. Cl. CPC C12Q 1/6883; C12Q 1/686; C12Q 2600/158; G01N 2800/368; C12Q 2561/113; G01N 2800/085; C12Q 2600/178

(57) ABSTRACT

A serum/plasma LncRNA marker composition associated with auxiliary diagnosis of intrahepatic cholestasis of pregnancy and application thereof. The marker is selected from any one of ENST00000449605.1, ASO3480 and ENST00000505175.1. The serum/plasma LncRNA screening and auxiliary diagnosis kit is a LncRNAs diagnostic kit developed based on the specific serum/plasma LncRNA of ICP cases and healthy controls.

This application claims priority to Chinese Patent Application Ser. No. CN201910132726.3 filed on 22 Feb. 2019.

### FIELD OF INVENTION

The invention belongs to the field of genetic engineering and reproductive medicine, and relates to a serum/plasma LncRNA marker composition associated with auxiliary diagnosis of intrahepatic cholestasis of pregnancy and application thereof.

### BACKGROUND ART

Intrahepatic cholestasis of pregnancy (ICP) is an idiopathic liver disease of pregnancy, which is extremely harmful to perinatal infants and clinically characterized by elevated bile acid, abnormal liver function, and itchy skin. The incidence of ICP is about 4.5%-15% in the world, 1%-4% in China's Yangtze River Basin and over 5% in both Chongqing and Chengdu. It has been reported that the incidence of ICP can cause poor prognosis of mother and infants including premature rupture of membranes, premature delivery, fecal amniotic fluid staining, intrauterine distress, unexplained death of the fetus, and postnatal hemorrhage of pregnant women. Various clinical treatments can only alleviate symptoms such as pruritus in pregnant women, but not reduce the harm to the fetus. Although it has been reported that many genes and proteins of ICP were changed, and may be involved in the occurrence of ICP through apoptosis, oxidative stress, lipid metabolism, cell growth, and immune response, the exact cause of ICP is still unknown.

Early diagnosis, early intervention and reasonable treatment of the ICP patients can effectively reduce the risk of the disease and complications of the mother and infant, and greatly reduce the pain and economic pressure caused by the ICP to the mother and child and their families. However, the current clinical monitoring methods of ICP are very limited and can only rely on the less sensitive bile acid screening. Therefore, the study of sensitive molecular events in ICP and the screening of susceptible biomarkers can provide effective means for the early diagnosis and intervention of ICP, which has great scientific significance for promoting human mother and infant health.

Long non-coding RNAs (lncRNAs) are a class of long endogenous non-coding RNAs with a length greater than 200 nucleotides (nt) and are highly conservative in evolution. Although initially thought to be RNAs which have no biological function and does not encode protein after transcription, it has been proven in recent years that it can be released by cells to the outside, exercise information transmission functions in different tissues and cells and plays an important biological role, and its function involves almost all areas of life. Studies have confirmed that LncRNA is more abundant in quantity, type, function and mode of action, and that LncRNAs in serum/plasma are stable in nature, abundant in content, easy to quantify, and have significant disease specificity. Existing mature technologies, including technology to qualify and quantify LncRNAs molecules, showed that the method of using serum LncRNAs as molecular biomarkers will be more effective than traditional specific protein molecular marker methods, opening up new possibilities for biomarkers.

However, there are no reports of more stable biomarkers for ICP-assisted diagnosis. If ICP-specific or abnormally expressed serum/plasma LncRNAs can be screened as biomarkers, and corresponding auxiliary diagnostic kits will be developed, it will greatly improve the diagnosis of ICP in China.

### SUMMARY OF INVENTION

The primary object of the present invention is to solve the above technical problems, and provides a serum/plasma LncRNA marker associated with ICP-assisted diagnosis.

A second object of the present invention is to provide primers for the above-mentioned serum/plasma LncRNA marker.

A third object of the present invention is to provide use of the serum/plasma LncRNA marker and its primers in the preparation of an ICP-assisted diagnostic kit.

A fourth object of the present invention is to provide a kit for ICP-assisted diagnosis.

The object of the present invention is achieved by the following technical solutions:

Provided is a serum/plasma LncRNA marker or a composition thereof associated with auxiliary diagnosis of intrahepatic cholestasis of pregnancy, the marker is selected from at least any one of ENST00000449605.1, ASO3480 and ENST00000505175.1. The cDNA sequence of ENST00000449605.1 is shown in SEQ ID No.7, the cDNA sequence of ASO3480 is shown in SEQ ID No.8, and the cDNA sequence of ENST00000505175.1 is shown in SEQ ID No.9.

Provided is a serum/plasma LncRNA marker associated with auxiliary diagnosis of intrahepatic cholestasis of pregnancy, the marker is selected from any one of ENST00000449605.1, ASO3480 and ENST00000505175.1.

Provided is a serum/plasma LncRNA marker composition associated with auxiliary diagnosis of intrahepatic cholestasis of pregnancy, preferably from any two of ENST00000449605.1, ASO3480 and ENST00000505175.1.

Provided is a serum/plasma LncRNA marker composition associated with auxiliary diagnosis of intrahepatic cholestasis of pregnancy, the marker preferably consists of three LncRNAs of ENST00000449605.1, ASO3480 and ENST00000505175.1.

Provided is a primer composition associated with auxiliary diagnosis of intrahepatic cholestasis of pregnancy, said primer or composition thereof is at least selected from primers that specifically amplify any one of ENST00000449605.1, ASO3480 and ENST00000505175.1.

The primer of ENST00000449605.1 in the primer composition is preferably as shown in SEQ ID No. 1 and SEQ ID No. 2; the primer of ASO3480 is preferably as shown in SEQ ID No. 3 and SEQ ID No. 4; the primers of ENST00000505175.1 are preferably as shown in SEQ ID No. 5 and SEQ ID No. 6.

The primer composition associated with auxiliary diagnosis of intrahepatic cholestasis of pregnancy may be primers that specifically amplify any one of ENST00000449605.1, ASO3480 and ENST00000505175.1.

The primer composition associated with auxiliary diagnosis of intrahepatic cholestasis of pregnancy is preferably consisted of primers that specifically amplify any two LncRNAs of ENST00000449605.1, ASO3480 and ENST00000505175.1.

The primer composition associated with auxiliary diagnosis of intrahepatic cholestasis of pregnancy is preferably composed of primers that specifically amplify three LncRNAs in ENST00000449605.1, ASO3480 and ENST00000505175.1.

provided is application of the serum/plasma LncRNA marker or a composition thereof as a detection target in preparing an auxiliary diagnosis kit for intrahepatic cholestasis of pregnancy.

provided is application of a reagent for detecting serum/plasma LncRNA markers or a composition thereof in the present invention in preparing an auxiliary diagnosis kit for intrahepatic cholestasis of pregnancy.

The reagent for detecting the serum/plasma LncRNA marker of the present invention is preferably the primer composition of the present invention.

Provided is an auxiliary diagnostic kit for intrahepatic cholestasis of pregnancy, the kit comprises a reagent for detecting serum/plasma LncRNA markers or a composition thereof according to the present invention.

The kit preferably contains a primer composition according to the present invention.

The kit may preferably further contain enzymes and reagents commonly used in PCR reactions.

The diagnostic kit may also contain enzymes and reagents commonly used in PCR reactions, such as reverse transcriptase, buffer, dNTPs, MgCl₂, DEPC water, and Taq enzymes, etc.; it may also contain standards and/or reference substances.

When the kit is a kit for detecting the serum/plasma LncRNA marker or a composition thereof based on the Taqman probe method, it should further include a Taqman probe for detecting the LncRNA marker or a composition thereof.

Specifically, the serum/plasma LncRNA markers of the present invention are obtained by screening according to the following methods: (1) creating a unified standard specimen library and database: collecting standard blood samples using standard operating procedures (SOP), and systematically collecting complete demographic and clinical data. (2) analyzing serum/plasma LncRNA differential expression profiles: selecting ICP cases, healthy female controls that match the age of ICP cases, detecting serum/plasma LncRNA expression profiles and contents of ICP cases and controls, and analyzing the commonness and differences of serum/plasma LncRNA between ICP cases and healthy female controls, screening differentially expressed LncRNAs for further multi-stage verification. (3) screening disease-specific serum/plasma LncRNAs: quantitative analyzing the screened differentially expressed serum/plasma LncRNAs in a large sample population to determine ICP-specific serum/plasma LncRNAs.

The serum/plasma LncRNA screening and auxiliary diagnosis kit of the present invention is a LncRNAs diagnostic kit developed based on the specific serum/plasma LncRNA of ICP cases and healthy controls.

The inventors used standard operating procedures (SOP) to collect standard blood samples, systematically collected complete demographic and clinical data (these data can be used to judge the impact of disease progression, patient age, and other factors on the incidence), and adopted RT-PCR, Real-time PCR method, CapitalBio Technology Human LncRNA Array v4 chip detection, etc.

Specifically, the experimental methods of study mainly include the following parts:

1. Selection of research samples

(1) The case group: ICP diagnostic criteria refer to the ICP patient diagnosis and treatment guidelines (first edition), the specific criteria were given as follows: 1) skin itch in the middle and late pregnancy, or accompanied by jaundice to varying degrees; 2) laboratory tests: serum total bile acid was elevated (TBA) (>40μmol/L), or accompanied by mild to moderate elevation of transaminase (ALT and AST), may be accompanied by elevated bilirubin; 3) pregnancy was the only cause of skin itch and biochemical abnormalities; 4) the patient was generally in good condition, without obvious symptoms of vomiting, poor appetite, weakness, and other diseases; 5) the above symptoms, signs, and serum biochemical indicators quickly returned to normal after delivery. 54 ICP patients with complete clinical data were collected.

(2) The normal control group: there are no pregnancy complications and comorbidities, and the indications for cesarean section were hip position, pelvic abnormality, and social factors. 54 normal pregnant women with complete clinical data were collected.

(3) Exclusion criteria of two groups: 1) having other hepatobiliary diseases; 2) having other pregnancy complications such as hypertension during pregnancy or having blood, urine or biochemical abnormalities that cannot be explained by ICP; 3) having systemic diseases such as diabetes, hypertension, mental and neurological diseases, etc.; 4) having genetic or immune diseases; 5) having history of blood transfusion, transplant or immunotherapy; 6) having history of oral contraceptives.

A total of 108 eligible samples were used for the study.

2. Trizol reagent (Invitrogen, USA) was used to extract serum/plasma total RNA, and the total RNA was further purified by column using NucleoSpin® RNA clean-up kit (740.948.250). According to routine operations, usually˜5 μg RNA/50 ml serum or plasma can be obtained.

3. Human LncRNA Array v4 (Capitalbio Technology) chip detection

(1) Obtaining cDNA samples by reverse transcription reaction of total RNA.

(2) Obtaining LncRNA expression profile through Human LncRNA Array v4 chip detection.

(3) Carrying out data analysis and processing.

4. Real-time RT-PCR (Q-PCR) method

(1) Taking the serum/plasma total RNA of the subject and obtaining a cDNA sample by RNA reverse transcription reaction;

(2) Designing primers;

(3) Quantitatively detecting LncRNA by Sybrgreen fluorescent dye method;

(4) Detecting and comparing changes in the amount of LncRNA in serum/plasma samples from ICP cases and healthy controls.

5. Preparation of diagnostic kit

The Human LncRNA Array v4 chip detection method was used to comprehensively determine the LncRNAs with differential expression in ICP cases and healthy controls, and a set of serum/plasma LncRNAs with large expression level and difference in ICP cases and healthy controls by Q-PCR technology was screened out as ICP Indicators of diagnosis. Finally, the serum/plasma LncRNAs associated with the pathogenesis of ICP resulted through screening were used to constitute the diagnostic kit (ENST00000449605.1, ASO3480 and ENST00000505175.1). The diagnostic kit included primers, probes, Taq enzymes, and dNTPs of these serum/plasma LncRNA compositions.

6. Statistical analysis methods

The student t test was used to compare the demographic characteristics, including the differences in the distribution of TAB (μmol/L), ALT (IU/L), AST (IU/L) and LncRNA average expression levels among the study groups.

In the exploratory sample population (4 ICP cases and 4 healthy controls), the Human LncRNA Array v4 chip was used to conduct preliminary screening and it was found that 58 LncRNAs were up-regulated and 85 LncRNAs were down-regulated. The correlation between the three differentially expressed LncRNAs (ENST00000449605.1, ASO3480 and ENST00000505175.1) and the incidence of ICP was then verified. Different expression levels detected by individual LncRNAs were expressed as 2^{-ΔΔCt}, wherein ΔCt=C_{T sample}−C_{T internal reference}, and miR-39 was used as the external reference gene to calculate the relative expression level. LncRNAs with statistically significant differences were further verified by Q-PCR in another 54 cases and 54 controls.

Statistical analysis was performed using SPSS16.0 statistical analysis software. The statistical significance level P value was set to 0.05, and all statistical tests were two-sided.

The invention is further described as follows:

In the above four eligible ICP cases and four healthy controls, the ages of the two groups were precisely matched according to individuals. We took these two groups of people as exploratory samples and tested them with Human LncRNA Array v4 chip to obtain relevant results.

According to the Human LncRNA Array v4 chip detection, the inventors detected LncRNA with differential expression (different expression (up-regulation or down-regulation) compared to the control group >2 times) in the serum of the “intrahepatic cholestasis of pregnancy cases” group and “healthy female controls” group) included: RNA95791|RNS_873_113, ENST00000584829.1, ENST00000446102.1, ENST00000523759.1, ENST00000534653.1, TCONS_00011955, TCONS_00009146, ENST00000449605.1, ENST00000439804.1, ENST00000604818.1, ENST00000609910.1, ENST00000600160. 1, ASO3480, ENST00000536898.1, HIT000430355, ENST00000483023.1, TCONS_00006708, HIT000248174, ENST00000505175.1, etc.

LncRNAs with a CT value of no more than 35 in two groups of human subjects in Human LncRNA Array v4 chip and a relatively uniform expression signal among the individual sample of each group were further verified by Q-PCR method to improve detection efficiency.

LncRNAs that met the above conditions included: ENST00000449605.1, ASO3480, and ENST00000505175.1.

Sybrgreen fluorescent dye method Q-PCR results showed that in 54 ICP cases and 54 healthy controls, 3 types of LncRNAs (ENST00000449605.1, ASO3480 and ENST00000505175.1) were found to have significant differences in expression in the “ICP case” group and the “health control” group.

Multivariate logistic regression analysis results showed that ENST00000449605.1, ASO3480 and ENST00000505175.1 were significantly associated with the pathogenesis of ICP and the composition of these three LncRNAs was more effective as a biomarker for ICP.

Based on the above experimental results, the present inventors prepared a kit that can be used for ICP-assisted diagnostics, including the primers for detection of ENST00000449605.1, ASO3480 and ENST00000505175.1 which were stably presented in serum/plasma of the subject and other test reagents. Specifically, the composition of these 3 types of LncRNAs, or related diagnostic kits composed of primers of these 3 types of LncRNAs, helps early diagnosis of ICP, provides timely support for accurate diagnosis of ICP, prevention and treatment protocols, thereby minimizing the risk of ICP leading to adverse pregnancy outcomes.

The beneficial effects of the present invention are given below:

The advantages of the serum/plasma long-chain non-coding RNA (LncRNAs) markers of the present invention as markers for ICP diagnosis are given below:

The inventors isolated and studied LncRNAs in serum/plasma from ICP cases of primiparous and singleton pregnancy and their age-matched healthy pregnant women controls to find a set of highly specific and sensitive LncRNAs highly associated with the pathogenesis of ICP, and developed ICP-assisted diagnostic kits for clinical applications to provide laboratory support for ICP screening and diagnosis and treatment.

The human LncRNA Array v4 chip detection was used to obtain the disease-specific and abnormally expressed serum/plasma LncRNAs expression profile in the early stage of the present invention, which was verified by the Q-PCR method using fluorescent dye method in the large samples; the application of the above methods and strategies accelerated and guaranteed the application of serum/plasma LncRNAs biomarkers and diagnostic kits, and also provided reference for the development of methods and strategies for other disease biomarkers.

The present invention studied the application prospects of serum/plasma LncRNAs in the diagnosis of ICP by controlling the factors that influence the development of the disease, such as age, elaborated the influence of abnormally expressed LncRNAs on the progress of ICP, and revealed its diagnostic value for ICP. Therefore, the present invention obtained an ICP pathogenesis-specific serum/plasma LncRNAs expression database and specific markers; the development and application of serum/plasma LncRNAs biomarkers and diagnostic kits made the diagnosis of ICP more convenient and easier, laid the foundation for clinicians to rapidly and accurately diagnose ICP and take therapeutic measures, and helped to discover new small molecule drug targets with potential therapeutic value.

### DESCRIPTION OF DRAWINGS

FIG. 1 Diagnostic value of serum LncRNA for ENST00000449605.1;

FIG. 2 Diagnostic value of serum LncRNA for ASO3480;

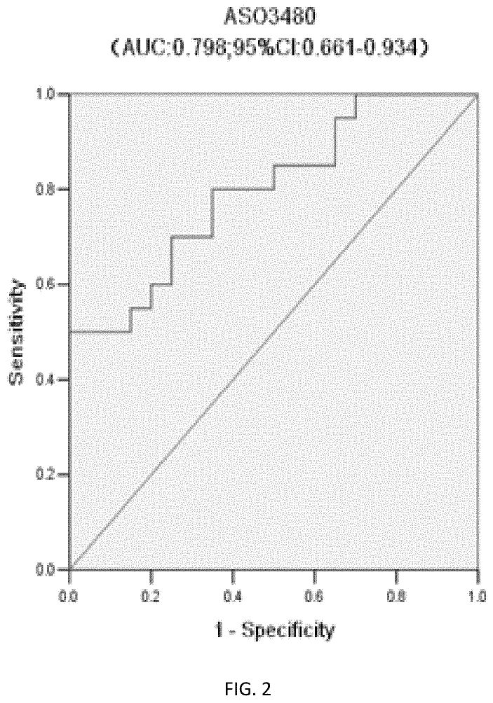

FIG. 3 Diagnostic value of serum LncRNA for ENST00000505175.1;

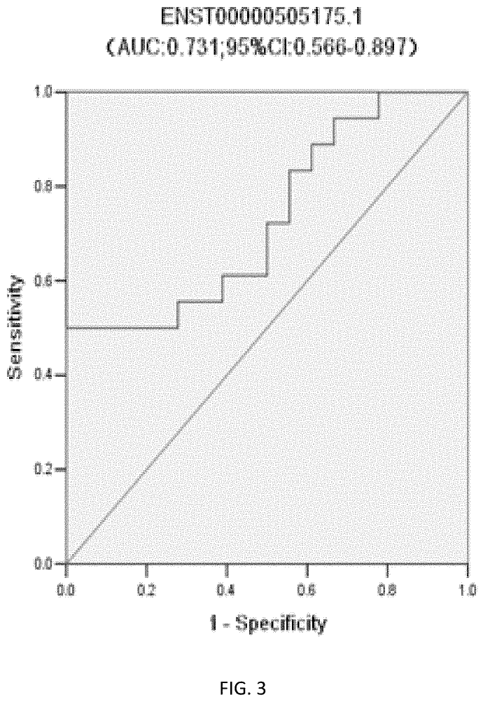

FIG. 4 Diagnostic value of serum LncRNA. ROC curve of a composition of three LncRNAs using multiple regression analysis;

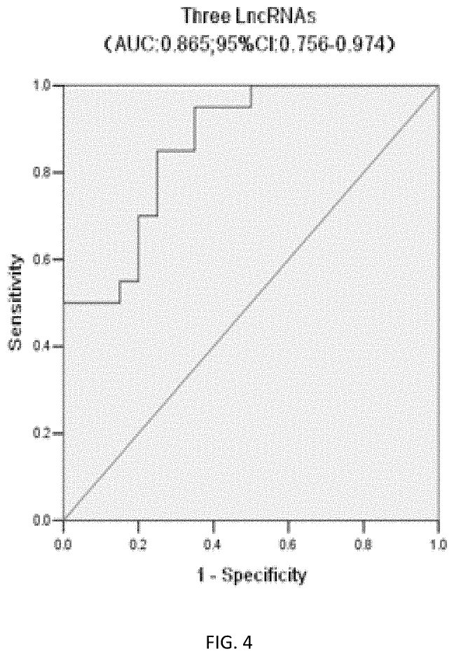

FIG. 5 Diagnostic value of serum LncRNA for ENC00000449605.1+ENST00000505175.1 combined ROC curve;

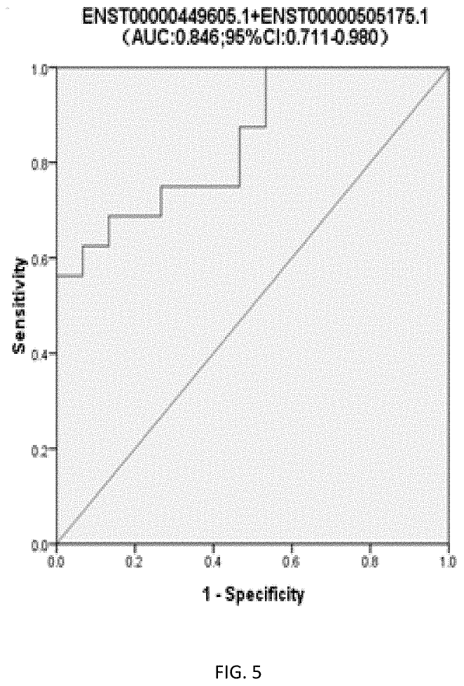

FIG. 6 Diagnostic value of serum LncRNA for ASO3480+ENST00000505175.1 combined ROC curve;

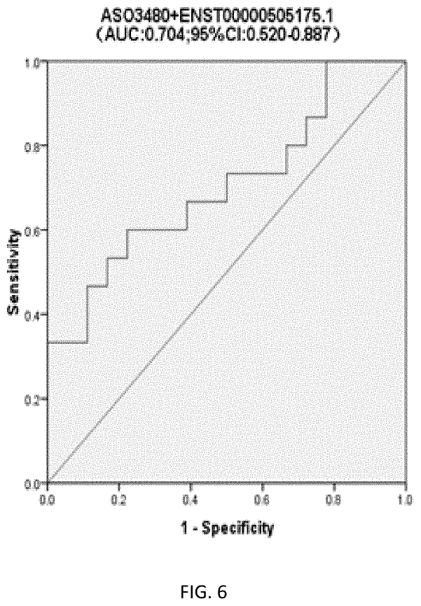

FIG. 7 Diagnostic value of serum LncRNA for ASO3480+ENST00000449605.1 combined ROC curve.

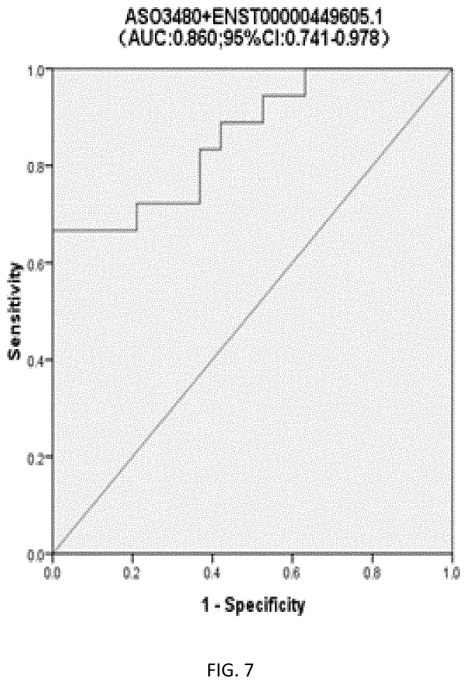

The composition of the three LncRNAs (ENST00000449605.1, ASO3480 and ENST00000505175.1) produces the largest area under the ROC curve (AUC).

### EMBODIMENTS FOR CARRYING OUT INVENTION

### Example 1 Collecting Samples and Collating Sample Data

The inventor collected a large number of peripheral blood samples of ICP patients and healthy control pregnant women from Wuxi Maternal and Child Health Hospital affiliated to Nanjing Medical University from October 2016 to September 2017(samples used for research were collected at the same time, and the conditions of sampling, packaging and storage were uniform). By collating the sample data, the inventors selected 108 samples that met the following criteria as experimental samples for human LncRNA Array v4 chip detection and subsequent series of Q-PCR verification:

1. The pregnant women in the above subjects who were confirmed as ICP during the ICP screening in the middle and late pregnancy (refer to the ICP Patient Diagnosis Guide (First Edition)) were defined as cases.

2. The healthy pregnant women in the above subjects who did not develop ICP during ICP screening in the middle and late pregnancy and who were matching the age and gestational age of the case group were defined as controls.

The demographic and clinical data of these samples were collected systematically.

### Example 2 Human LncRNA Array v4 Chip Detection of LncRNAs in Serum/Plasma

The above 4 eligible ICP cases and 4 healthy controls were detected by Human LncRNA Array v4 chip to obtain relevant results. The specific steps were given below:

1. Extraction and quality check of total RNA

Appropriate method, such as Trizol (Invitrogen, USA) was used to extract total RNA from tissue blocks or cells. The total RNA was further purified by column using the NucleoSpin® RNA clean-up kit (740.948.250). Then a spectrophotometer or Qubit was used to quantify, and agarose gel electrophoresis or Agilent 2100 was used to check its integrity.

2. Synthesis of cDNA by total RNA 2.1 Synthesis of First Strand cDNA by Reverse Transcription

The following reagents were added to a 0.2 mL of nuclease-free centrifuge tube:

2.1.1 5 μL of Total RNA (100-500 ng) was added to a 0.2 mL of nuclease-free centrifuge tube.

2.1.2 The corresponding volume of Agilent spike-in was added. 2.0 μL of Spike A (Agilent) or 2.0 μL of Spike B (Agilent) was added to all samples.

2.1.3 The reverse transcription Master Mix was prepared on ice, gently mixed, briefly centrifuged and placed on the ice bath.

2.1.4 5 μL of the reverse transcription Master Mix was added to a 0.2 mL of centrifuge tube containing Total RNA samples. The final reaction volume for reverse transcription was 10 μL.

2.1.5 The centrifuge tube was mixed by pipetting for 2-3 times, immediately centrifuged and placed on ice.

2.1.6 The reverse transcription centrifuge tube was placed on the PCR instrument, reacted at 25° C. for 1 hour, reacted at 42° C. for 1 hour, and kept at 4° C. for more than 5 minutes. The reverse transcription centrifuge tube was taken out, instantaneously centrifuged, and placed on ice for the Second Strand cDNA synthesis reaction.

2.2 Synthesis of Second Strand cDNA 2.2.1 The Second Strand Master Mix was prepared on ice, mixed gently, centrifuged briefly, and kept in an ice bath.

2.2.2 50 μL of Second Strand Master Mix was added to the reaction tube in step 2.1.6, with a mixing volume of 60 μL; mixed by pipetting for 2-3 times, centrifuged immediately, and placed on ice.

2.2.3 The second-strand synthesis centrifuge tube was placed on the PCR instrument, and reacted at 16° C. for 1 hour (turn off the lid heating function of the PCR instrument), reacted at 65° C. for 10 minutes, and kept at 4° C. for more than 5 minutes.

2.2.4 After the reaction, the reaction tube was placed on ice to continue the synthesis reaction, or quickly frozen at −20° C.

3. Synthesis of cRNA by in vitro transcription

3.1 Synthesis of cRNA

3.1.1 In vitro transcription Master Mix was prepared, mixed gently, and the solution after brief centrifugation was collected at the bottom of the tube.

3.1.2 30 μL of IVT Master Mix was added to the reaction tube in step 2.2.4, mixed by pipetting, and then placed on ice after instant centrifugation.

3.1.3 The in vitro transcription synthesis centrifuge tube was placed on the PCR instrument, and reacted at 40° C. for 16 h, and maintained at 4° C.

3.1.4 After the reaction, the cells were centrifuged instantaneously, and the products were purified using NucleoSpin® RNA clean-up kit (MN company, 740.948.250), and the purified cRNA products were quantified using a UV spectrophotometer.

4. cRNA reverse transcription

4.1 Synthesis of cDNA by cRNA reverse transcription

4.1.1 10 μg of the purified cRNA product was adjusted to the volume of 22 μL, and added to a 0.2 mL nuclease-free centrifuge tube, 2 μL of Random Primer was added, mixed thoroughly and placed on a PCR instrument, at 70° C. for 5 min, at 25° C. for 5 min, at 4° C. for 2 min. The liquid was collected at the bottom of the tube after instant centrifugation, and placed on ice.

4.1.2 cRNA reverse transcription Master Mix was prepared and mixed gently, and the solution after brief centrifugation was collected at the bottom of the tube.

4.1.3 16 μL of the reverse transcription Master Mix was added to the centrifuge tube after the reaction in step 4.1.1 with the total volume of 40 μL, mixed by pipetting for 2-3 times, and centrifuged immediately.

4.1.4 The cRNA reverse transcription centrifuge tube was placed on a PCR instrument, reacted at 25° C. for 10 minutes, 40° C. for 1.5 hours, 70° C. for 10 minutes, 4° C. for 5 minutes, and placed on ice.

4.1.5 Operations were done on ice. 2 μL of RNase H was added to the cRNA reverse transcription centrifuge tube, mixed thoroughly and centrifuged instantly, and the centrifuge tube was placed on a PCR instrument, reacted at 37° C. for 45 min, 95° C. for 5 min, and maintained at 4° C. for 5 min.

4.1.6 After the reaction, the product can be frozen at −20°C. overnight, or purified immediately.

4.1.7 Nucleospin® Extract II (MN, Cat. No. 740609.250) kit was used for cDNA purification, and the purified cRNA product was quantified using a UV spectrophotometer.

5. Fluorescent labeling

5.1 Fluorescent dye labeling reaction

5.1.1 The volume of the cDNA product obtained after reverse transcription and purification was concentrated to 14 μL, and 4 μL of Random Primer was added to mix thoroughly, which, after a brief centrifugation, was placed on a PCR instrument, denatured at 95° C. for 3 minutes, and ice-bathed for 5 minutes.

5.1.2 Relevant reagents were added one by one and mixed by pipetting for 2-3 times.

5.1.3 After a brief centrifugation, the product was placed on a PCR instrument, reacted at 37° C. for 1.5 hours, 70° C. for 5 minutes and maintained at 4° C.

5.1.4 After the fluorescent dye labeling reaction was completed, Nucleospin® Extract II (MN Co., Cat. No. 740609.250) kit was used for cDNA purification, and a UV spectrophotometer was used to perform fluorescence incorporation and nucleic acid quantification on the purified fluorescently labeled product.

6. Chip hybridization

6.1 Preparation of labeled products for hybridization

6.1.1 The eluted volume of the labeled product purified by Nucleospin® Extract II kit was about 30 μL.

6.1.2 The single-tube labeled cy3-dCTP purified eluted product was concentrated under vacuum or filled with water to a volume of 27.5 μL for later use.

6.2 Hybridization system preparation and hybridization reaction

6.2.1 The labeled product prepared in step 6.1 was mixed with the corresponding reagents.

6.2.2 100 μL of hybridization solution was added to the hybridization cover fence, the fence was covered with the “Agilent” label face down, the Agilent hybridization box was installed and screwed tightly. Then, the hybridization box can be gently rotated horizontally to check whether the liquid in the hybridization chamber of each array was flowing.

6.2.3 The hybridization box was installed on the rotor of the hybridization furnace and symmetrical installation was necessary. At the same time, an appropriate amount of ultrapure water was added to the tray, followed by hybridizing at 45° C. overnight.

7. Chip cleaning and scanning

7.1 After the hybridization was over, the chips were taken out and washed in the Boao Slide Washer 8 chip washer-dryer. The cleaning procedure was given below:

Washing solution I: 0.2% SDS, 2×SSC, and 120° C., wash twice. Wash solution II: 0.2% SDS, 2×SSC, and 42° C. 80S, wash 3 times. After the cleaning procedure was completed, drying by centrifuge was done for scanning.

7.2 The cleaned chip was washed with an Agilent chip scanner (G2565CA) to obtain hybrid images.

8. Chip data analysis

The Agilent Feature Extraction (v10.7) software was used to analyze the hybrid images and extract the data. The data was then subject to normalization and variance analysis using Agilent GeneSpring software.

In the 4 ICP cases and 4 healthy controls, the Human LncRNA Array v4 chip was used to conduct preliminary screening and 58 LncRNAs up-regulation and 85 LncRNAs down-regulation were found, specifically including: RNA95791|RNS_873_113, ENST00000584829.1, ENST00000446102.1, ENST00000523759.1, ENST00000534653.1, TCONS_00011955, TCONS_00009146, ENST00000449605.1, ENST00000439804.1, ENST00000604818.1, ENST00000609910.1, ENST00000600160.1, ASO3480, ENST00000536898.1, HIT000430355, ENST00000483023.1, TCONS_00006708, HIT000248174, ENST00000505175.1, etc.

### Example 3 Q-PCR Experiment of LncRNA in Serum/Plasma

LncRNAs with a CT value no more than 35 in two groups of subjects in Human LncRNA Array v4 chip and a relatively uniform expression signal among the individual sample of each group were further verified by Q-PCR method to improve detection efficiency.

LncRNAs that meet the above conditions included: ENST00000449605.1, ASO3480, and ENST00000505175.1.

Based on the above Human LncRNA Array v4 results, LncR-371a-5p, LncR-6865-5p, LncR-1182, ENST00000449605.1, ASO3480 and ENST00000505175.1 were selected to design primers for reverse transcription and Q-PCR, as shown in Table 1. Q-PCR detection of LncRNA was performed on individual serum of the “ICP case” group and the “healthy control” group. The results were shown in Table 2.

TABLE 1
Primer sequence

| | Upstream | downstream |
|---|---|---|
| | primer | primer |
| LncRNA | (5′-3′) | (5′-3′) |
| ENST00000449605.1 | CAGGCTGGGC | CCTGGGCTCA |
| | AACATAGTGA | AACGATGCT |
| | (SEQ ID | (SEQ ID |
| | No. 1) | No. 2) |
| ASO3480 | TTGATGGCTG | CCATGTTGAG |
| | GCAGTGCTC | GCAGCACATC |
| | (SEQ ID | (SEQ ID |
| | No. 3) | No. 4) |
| ENST00000505175.1 | GGCCAGTGAC | TTGCTGCCTC |
| | CTTGACCTT | TTATGCTCAC |
| | (SEQ ID | (SEQ ID |
| | No. 5) | No. 6) |

TABLE 2
Differential expression of LncRNA in ICP group and control group
LncRNA array (partial)

| IncRNA ID | FC (abs) | P value | Regulation |
|---|---|---|---|
| RNA95791\|RNS_873_113 | 6.964221431 | 0.001058622 | up |
| ENST00000584829.1 | 6.55149382 | 0.030199938 | down |
| ENST00000446102.1 | 6.403015575 | 4.2906E−05 | up |
| ENST00000523759.1 | 6.0832106 | 0.01107794 | down |
| ENST00000534653.1 | 5.871339591 | 0.000239719 | up |
| TCONS_00011955 | 5.860672153 | 0.033388355 | down |
| TCONS_00009146 | 4.175216237 | 0.035598435 | down |
| ENST00000449605.1 | 4.125941729 | 0.045659168 | down |
| ENST00000439804.1 | 4.076246383 | 0.020564769 | up |
| ENST00000604818.1 | 5.186548135 | 0.02255008 | down |
| ENST00000609910.1 | 5.148031753 | 0.020427679 | up |
| ENST00000600160.1 | 3.756218881 | 0.02171473 | down |
| ASO3480 | 3.743970405 | 0.045791717 | down |
| ENST00000536898.1 | 5.053739332 | 0.038369709 | down |
| HIT000430355 | 5.010350662 | 0.026398479 | down |
| ENST00000483023.1 | 4.959201157 | 0.007267478 | up |
| TCONS_00006708 | 2.964524124 | 0.049289246 | down |
| HIT000248174 | 3.148164318 | 0.048432019 | down |
| ENST00000505175.1 | 3.096611325 | 0.02923292 | down |

ICP (P) group had more than 2.0 times up-regulated or down-regulated LncRNAs than healthy pregnant women (C) group.

Strict quality control was implemented throughout the research process. Each sample was tested three times in a row. Blind method was used in all tests, i.e., the tests were finished without information of the background of the samples, to avoid bias. LncRNA quantitative detection was performed using the Sybrgreen fluorescent dye method.

1. RNA extraction

1.1 300 ul of sample was transferred to a 1.5 mL of centrifuge tube;

1.2 1000 μL of TRIzol LS® Reagent was added to a 1.5 mL of centrifuge tube, mixed with vigorous shaking, and maintained at room temperature for 10 mins.

1.3 200 μL of chloroform was added, mixed thoroughly with vortex shaking for 10 seconds, and maintained at room temperature for 5 mins.

1.4 The sample was placed in a centrifuge and centrifuged at 4° C., 12000 rpm for 15 mins.

1.5 The supernatant was transferred to a new 1.5 mL of centrifuge tube, an equal volume of isopropanol was added thereto, and the centrifuge tube was gently turned upside down until no filaments were visible, followed by maintaining at −20° C. for 30 mins.

1.6 The centrifuge tube was placed in a centrifuge, centrifuged at 4° C., 12000 rmp for 10 mins, and the supernatant was discarded.

1.7 1000 μL of 75% ice-cold ethanol was added, washed upside down, put it in a centrifuge, and centrifuge at 12,000 rpm at 4° C. for 10 min. Discard the supernatant.

1.8 After the precipitate is dried, add an appropriate amount of DEPC-H20 to dissolve it according to the amount of precipitation. Store at −80° C.

2. Reverse Transcription

A sterilized RNase-free Eppendorf tube was taken and the following components were added to each sample to obtain Mix I.

| Components | Sample amount (L) | Supplier |
|---|---|---|
| Up to 1 μg Total RNA | X | |
| Primer (10 μM) | 1.0 | random primer |
| dNTP Mix (10 mM) | 1.0 | Thermo Fisher |
| DEPC-treated water | 10-X | Thermo Fisher |
| Total | 12.0 | |

Mix I was incubated at 65° C. for 5 minutes, then immediately put on ice for 1 minute.

| Components | Sample amount (L) | Supplier |
|---|---|---|
| 5 × First-Strand Buffer | 4.0 | Thermo Fisher |
| 0.1M dTT | 2.0 | Thermo Fisher |
| RNaseout 40 U/μL | 1.0 | Thermo Fisher |
| SuperScrip III RT (200 U/μL) | 1.0 | Thermo Fisher |
| Mix I | 12.0 | |
| Total | 20.0 | |

The following components were added to Mix I. A total of 20 μL of Mix II was obtained.

The Mix II was:

treated at 25° C. for 5 minutes,

treated at 42° C. for 60 minutes,

treated at 70° C. for 15 minutes, and immediately placed on ice.

The obtained cDNA can be stored at −20° C. for half a year.

3. QPCR amplification

The volume of each component in the QPCR system:

| Components | Sample amount (L) | Supplier |
|---|---|---|
| 10 × PCR Buffer(−) | 2.0 | Thermo Fisher |
| Mg²⁺ (50 mM) | 1.0 | Thermo Fisher |
| dNTPs (10 mM) | 0.5 | Thermo Fisher |
| Forward Primer (10 · M) | 0.5 | |
| SYBR (20×) | 0.3 | Thermo Fisher |
| Reverse Primer (10 · M) | 0.5 | |
| Platinum ® Taq DNA | 0.2 | Thermo Fisher |
| Polymerase (5 U/L) | | |
| Template | 1.0 | |
| ddH₂O | 14.0 | Thermo Fisher |

QPCR reaction conditions:

| Stage | temperature | time | | Cycle amount | |
|---|---|---|---|---|---|
| Holding Stage | 95° C. | 60 | s | 1 | cycles |
| | 95° C. | 5 | sec | | |
| Cycling Stage | 60° C. | 40 | sec | 40 | cycles |

4. Data processing and analysis

LncRNA quantitative PCR detection data was processed using the comparative CT (ΔΔCT) method to calculate the fold change of relative gene expression. The data processing process mainly included:

The expression ratio of serum LncRNAs in the two groups of samples can be expressed by Equation 2^{-ΔCt}, wherein ΔCt=C_{T sample}−C_{T internal reference}, miR-39 as an external reference, and the relative expression (miR-39: SEQ ID No. 7 and SEQ ID No.8) was calculated.

Sybrgreen fluorescent dye method Q-PCR results showed that in 108 samples, the expression of three LncRNAs (ENST00000449605.1, ASO3480 and ENST00000505175.1) was significantly different between the two groups.

TABLE 3
Verification of sample expression results

| | ICP (n = 54) | | Control (n = 54) | | |
|---|---|---|---|---|---|
| LncRNAs | mean | SD | mean | SD | P value |
| ENST00000449605.1 | 20.01 | 0.2945 | 18.94 | 0.3376 | 0.0224 |
| ASO3480 | 23.28 | 0.2976 | 22.34 | 0.2436 | 0.0203 |
| ENST00000505175.1 | 3.746 | 0.0386 | 2.726 | 0.2797 | 0.0196 |

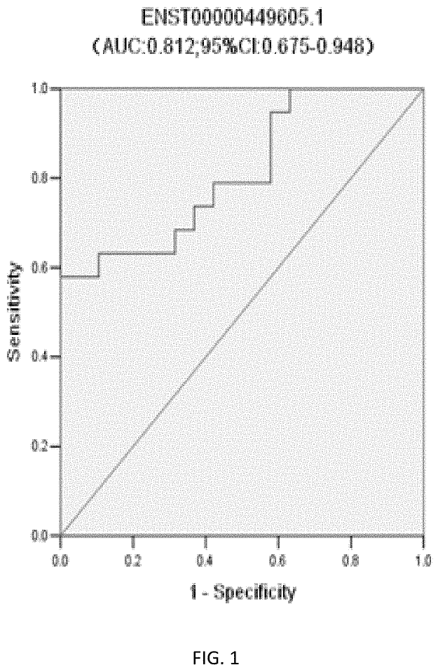

Multivariate logistic regression analysis results showed that ENST00000449605.1, ASO3480 and ENST00000505175.1 were significantly correlated with the pathogenesis of ICP. The composition of these three LncRNAs was more effective as a biomarker for ICP (FIG. 1).

### Example 4 Preparation of LncRNA Kit for ICP-Assisted Early Diagnosis

The preparation and operation process of LncRNA kit was based on Human LncRNA Array v4 chip detection, RT-PCR, QPCR and other technologies. The kit included serum/plasma LncRNA primers (including the following primers: the primers for ENST00000449605.1 were SEQ ID No. 1 and SEQ ID No. 2; the primers for ASO3480 were SEQ ID No. 3 and SEQ ID No. 4; the primers of ENST00000505175.1 were SEQ ID No. 5 and SEQ ID No. 6), and can also have common enzymes and/or reagents required for corresponding PCR reactions, such as: reverse transcriptase, buffer, dNTPs, MgCl₂, denuclease water, fluorescence dyes or probes, Taq enzymes, etc., which can be selected according to the specific experimental method used, these commonly used enzymes and/or reagents are well known to those skilled in the art, and standards and controls (such as quantitatively standardized nematode LncR-39 samples, etc.) and normal reference values can also be included. The value of this kit was that it only required serum/plasma and no other tissue samples, which could detect the change trend of LncRNA by the most simplified fluorescence or probe method and use this trend to assist early diagnosis of ICP. The kit, which was not only stable, convenient to detect, but also quantitative and accurate, can greatly improve the sensitivity and specificity of disease diagnosis. Therefore, putting this kit into practice can help guide accurate clinical diagnosis.

### Example 5 Preparation of LncRNA Kit for ICP-Assisted Early Diagnosis

The preparation and operation process of LncRNA kit was based on Human LncRNA Array v4 chip detection, RT-PCR, QPCR and other technologies. The kit included serum/plasma LncRNA primers (including the following primers: the primers for ENST00000449605.1 were SEQ ID No. 1 and SEQ ID No. 2; the primers for ASO3480 were SEQ ID No. 3 and SEQ ID No. 4), and can also have common enzymes and/or reagents required for corresponding PCR reactions, such as: reverse transcriptase, buffer, dNTPs, MgCl₂, denuclease water, fluorescence dyes or probes, Taq enzymes, etc., which can be selected according to the specific experimental method used, these commonly used enzymes and/or reagents are well known to those skilled in the art, and standards and controls (such as quantitatively standardized nematode LncR-39 samples, etc.) and normal reference values can also be included. The value of this kit was that it only required serum/plasma and no other tissue samples, which could detect the change trend of LncRNA by the most simplified fluorescence or probe method and use this trend to assist early diagnosis of ICP. The kit, which was not only stable, convenient to detect, but also quantitative and accurate, can greatly improve the sensitivity and specificity of disease diagnosis. Therefore, putting this kit into practice can help guide accurate clinical diagnosis.

### Example 6 Preparation of LncRNA Kit for ICP-Assisted Early Diagnosis

The preparation and operation process of LncRNA kit was based on Human LncRNA Array v4 chip detection, RT-PCR, QPCR and other technologies. The kit included serum/plasma LncRNA primers (including the following primers: the primers for ASO3480 were SEQ ID No. 3 and SEQ ID No. 4; the primers of ENST00000505175.1 were SEQ ID No. 5 and SEQ ID No. 6), and can also have common enzymes and/or reagents required for corresponding PCR reactions, such as: reverse transcriptase, buffer, dNTPs, MgCl₂, denuclease water, fluorescence dyes or probes, Taq enzymes, etc., which can be selected according to the specific experimental method used, these commonly used enzymes and/or reagents are well known to those skilled in the art, and standards and controls (such as quantitatively standardized nematode LncR-39 samples, etc.) and normal reference values can also be included. The value of this kit was that it only required serum/plasma and no other tissue samples, which could detect the change trend of LncRNA by the most simplified fluorescence or probe method and use this trend to assist early diagnosis of ICP. The kit, which was not only stable, convenient to detect, but also quantitative and accurate, can greatly improve the sensitivity and specificity of disease diagnosis. Therefore, putting this kit into practice can help guide accurate clinical diagnosis.

### Example 7 Preparation of LncRNA Kit for ICP-Assisted Early Diagnosis

The preparation and operation process of LncRNA kit was based on Human LncRNA Array v4 chip detection, RT-PCR, QPCR and other technologies. The kit included serum/plasma LncRNA primers (including the following primers: the primers for ENST00000449605.1 were SEQ ID No. 1 and SEQ ID No. 2; the primers of ENST00000505175.1 were SEQ ID No. 5 and SEQ ID No. 6), and can also have common enzymes and/or reagents required for corresponding PCR reactions, such as: reverse transcriptase, buffer, dNTPs, MgCl₂, denuclease water, fluorescence dyes or probes, Taq enzymes, etc., which can be selected according to the specific experimental method used, these commonly used enzymes and/or reagents are well known to those skilled in the art, and standards and controls (such as quantitatively standardized nematode LncR-39 samples, etc.) and normal reference values can also be included. The value of this kit was that it only required serum/plasma and no other tissue samples, which could detect the change trend of LncRNA by the most simplified fluorescence or probe method and use this trend to assist early diagnosis of ICP. The kit, which was not only stable, convenient to detect, but also quantitative and accurate, can greatly improve the sensitivity and specificity of disease diagnosis. Therefore, putting this kit into practice can help guide accurate clinical diagnosis.

### Example 8 Preparation of LncRNA Kit for ICP-Assisted Early Diagnosis

The preparation and operation process of LncRNA kit was based on Human LncRNA Array v4 chip detection, RT-PCR, QPCR and other technologies. The kit included serum/plasma LncRNA primers (including any one group of the following primers: The primers for ENST00000449605.1 were SEQ ID No. 1 and SEQ ID No. 2; the primers for ASO3480 were SEQ ID No. 3 and SEQ ID No. 4; the primers of ENST00000505175.1 were SEQ ID No. 5 and SEQ ID No. 6), and can also have common enzymes and/or reagents required for corresponding PCR reactions, such as: reverse transcriptase, buffer, dNTPs, MgCl₂, denuclease water, fluorescence dyes or probes, Taq enzymes, etc., which can be selected according to the specific experimental method used, these commonly used enzymes and/or reagents are well known to those skilled in the art, and standards and controls (such as quantitatively standardized nematode LncR-39 samples, etc.) and normal reference values can also be included. The value of this kit was that it only required serum/plasma and no other tissue samples, which could detect the change trend of LncRNA by the most simplified fluorescence or probe method and use this trend to assist early diagnosis of ICP. The kit, which was not only stable, convenient to detect, but also quantitative and accurate, can greatly improve the sensitivity and specificity of disease diagnosis. Therefore, putting this kit into practice can help guide accurate clinical diagnosis.

What is claimed is:

1. A diagnostic kit for detecting an intrahepatic cholestasis of pregnancy in a sample of a subject comprising a reverse transcriptase, a DNA polymerase, dNTPs, a buffer and at least one pair of primers capable of amplifying one fragment of DNA from cDNA of an ENST00000449605.1, ASO3480 or ENST00000505175.1.

2. The diagnostic kit according to claim 1, wherein the ENST00000449605.1, ASO3480 and ENST00000505175.1 are RNAs whose cDNAs having DNA sequences shown as SEQ ID NO: 7, SEQ ID NO: 8 and SEQ ID NO: 9, respectively.

3. The diagnostic kit according to claim 2, wherein the pair of primers has the DNA sequences shown in SEQ ID No. 1 and SEQ ID No. 2, in SEQ ID No. 3 and SEQ ID No. 4 or in SEQ ID No. 5 and SEQ ID No. 6 which amplifies the fragment of DNA from the cDNA of the ENST00000449605.1, ASO3480 or ENST00000505175.1, respectively.

4. The diagnostic kit according to claim 1, wherein the buffer contains SYBR Green.

5. The diagnostic kit according to claim 1, wherein the kit is capable of using for real-time PCR.

6. The diagnostic kit according to claim 1, wherein the subject is a pregnancy woman, the sample is a serum or plasma from the pregnancy woman.

7. The diagnostic kit according to claim 1, wherein the kit comprises an instruction for using the kit to evaluate the intrahepatic cholestasis of pregnancy in the subject.

8. A method for detecting an intrahepatic cholestasis of pregnancy comprising a step for detecting a LncRNA (Long non-coding RNA) marker in a sample of a subject, the LncRNA marker is ENST00000449605.1, ASO3480 or ENST00000505175.1.

9. The method according to claim 8, wherein the ENST00000449605.1, ASO3480 and ENST00000505175.1 are RNAs whose cDNAs having DNA sequences shown as SEQ ID NO: 7, SEQ ID NO: 8 and SEQ ID NO: 9, respectively.

10. The method according to claim 8, wherein the method is a PCR-based reaction that includes a reverse transcriptase, a DNA polymerase, dNTPs, a buffer and at least one pair of primers capable of amplifying one fragment of DNA from the cDNA of the ENST00000449605.1, ASO3480 and ENST00000505175.1.

11. The method according to claim 10, wherein the pair of primers has the DNA sequences shown in SEQ ID No. 1 and SEQ ID No. 2, in SEQ ID No. 3 and SEQ ID No. 4 or in SEQ ID No. 5 and SEQ ID No. 6 which amplifies the fragment of DNA from the cDNA of the ENST00000449605.1, ASO3480 or ENST00000505175.1, respectively.

12. The method according to claim 8, wherein the subject is a pregnancy woman, the sample is a serum or plasma from the pregnancy woman.

13. The method according to claim 10, wherein comprises a step of analyzing results of the PCR-based reaction and evaluating the intrahepatic cholestasis of pregnancy in the subject.
